(19) United States
(12) Patent Application Publication
(10) Pub. No.: US 2021/0169559 A1
(43) Pub. Date: Jun. 10, 2021

(54) ACOUSTIC MONITORING FOR ELECTROSURGERY

(71) Applicant: Board of Regents, The University of Texas System, Austin, TX (US)

(72) Inventors: Wei Li, Austin, TX (US); Xiaoran Li, Austin, TX (US)

(21) Appl. No.: 17/112,337

(22) Filed: Dec. 4, 2020

Related U.S. Application Data
(60) Provisional application No. 62/944,736, filed on Dec. 6, 2019.

(51) Int. Cl. A61B 18/14; A61B 18/18; A61B 18/20

(52) U.S. Cl. CPC A61B 18/1445; A61B 2018/00577; A61B 18/20; A61B 18/1815

(57) ABSTRACT

The present invention provides devices and methods for improved tissue hemostasis and electrosurgery. The devices and methods are based on measuring acoustic signals near an electrosurgery site to determine when sufficient hemostasis is achieved. The devices include cautery devices and systems incorporating acoustic sensors and acoustic signal software.

### CROSS-REFERENCE TO RELATED APPLICATIONS

This application claims priority to U.S. Provisional Patent Application No. 62/944,736, filed on Dec. 6, 2019, incorporated herein by reference in its entirety.

### STATEMENT REGARDING FEDERALLY SPONSORED RESEARCH OR DEVELOPMENT

This invention was made with government support under Grant no. CMMI1434584 awarded by the National Science Foundation. The government has certain rights in the invention.

### BACKGROUND OF THE INVENTION

Bipolar tissue hemostasis, also known as tissue welding, is an electrosurgical operation that is performed with bipolar forceps where high frequency alternating current is used to seal vessels and stop bleeding in surgery. The advantages of bipolar tissue hemostasis compared to traditional hemostatic procedures include, but not limited to, less bleeding, shorter post-surgery recover time, and suitability for laparoscopic surgery (Bulsara K R et al., Neurosurgical review, 2006, 29(2):93-96; Kusunoki Metal., Diseases of the colon & rectum, 1998, 41(9):1197-1200; Law K S K et al., Journal of minimally invasive gynecology, 2013, 20(3):308-318; Munro M G, The SAGES Manual on the Fundamental Use of Surgical Energy (FUSE), 2012, 15-59; Vilos G A et al., Journal of minimally invasive gynecology, 2013, 20(3):279-287; Wang K et al., International Journal of Gynecology & Obstetrics, 2007, 97(3):245-250). Despite all the advantages, there is a lack of reliable monitoring methods to indicate whether acceptable hemostasis is achieved and when the heating power should be terminated (Campbell P A et al., Surgical Endoscopy and Other Interventional Techniques, 2003, 17(10):1640-1645; Bergdahl B et al., Journal of neurosurgery, 1991, 75(1):148-151; Keshavarzi S et al., World neurosurgery, 2015, 83(3):376-381). There are still major concerns such as tissue sticking and excessive thermal damage, some which could lead to fatal complications. Anti-sticking electrodes and irrigation have been used to mitigate these problems. However, they have only achieved limited success. A reliable monitoring method that can indicate whether acceptable hemostasis has been achieved and when the heating power should be terminated could be the key to solving the current problems associated with bipolar surgeries.

Monitoring the bipolar tissue hemostasis process initially relied on visual inspection by surgeons. The surgeon's view of the weld site is often limited by the laparoscope when performing minimally invasive surgeries. The weld site can also be easily buried in bodily fluids and the smoke generated during the surgery. Machine assisted monitoring methods, including impedance monitoring and temperature sensing, have been developed for improving the quality of bipolar tissue hemostasis (Campbell P A et al., Surgical Endoscopy and Other Interventional Techniques, 2003, 17(10):1640-1645; Bergdahl B et al., Journal of neurosurgery, 1991, 75(1):148-151; Vällfors B et al., Neurosurgical review, 1984, 7(2-3):185-189; Cezo J D et al., Journal of the mechanical behavior of biomedical materials, 2014, 30:41-49). However, these monitoring methods cannot guarantee the outcome of joint quality. The minimal impedance monitoring method was introduced in the 1980s and has been used by certain equipment vendors as a criterion to stop the heating power. The dynamic impedance during the heating process decreases initially due to the enlarged contact area between the electrodes and the tissue. With elevated temperature, the impedance will increase due to tissue desiccation. This characteristic has been used as a criterion to stop the heating process. However, it has been shown that the impedance measurement can be affected by many factors such as welding site irrigation, changing displacement between electrodes, and various power settings of the bipolar hemostasis process. The initial impedance measurement between the electrodes has also been used to determine the amount of energy to be delivered to the tissue. However, these pre-calibrated generators are extremely sensitive to tissue compression, electrode coating, and residual tissue sticking conditions, which strongly affect the initial impedance measurement. As a result, in current bipolar electrosurgical operations, side effects such as tissue sticking, charring, and excessive thermal damage often occur (Mikami T et al., Journal of neurosurgery, 2004, 100(1):133-138; Rondinone J et. al., Surgical Applications of Energy, 1998, 3249:142-147; Campbell P A et al., Surgical Endoscopy and Other Interventional Techniques, 2003, 17(10):1640-1645; Chen R K et al., Surgical neurology international, 2013, 4; Phillips C K et al., Urology, 2008, 71(4):744-748), sometimes leading to fatal complications under extreme circumstances (Chen R K et al., IEEE Trans. Biomed. Engineering, 2013, 60(2):453-460; Fuller A et al., Photonic Therapeutics and Diagnostics VIII, 2012, 8207).

There is a need in the art for improved devices and methods for tissue hemostasis and cauterization. The present invention meets this need.

### SUMMARY OF THE INVENTION

In one aspect, an electrosurgery device comprises at least one ablative element; at least one acoustic sensor; and at least one power lead connected to the at least one ablative element; wherein the at least one microphone is positioned at a distance from the at least one ablative element.

In one embodiment, the at least one ablative element is selected from the group consisting of: bipolar electrode forceps, unipolar electrodes, laser probes, radiofrequency probes, and microwave probes. In one embodiment, the bipolar electrode forceps are laparoscopic forceps or tweezer forceps. In one embodiment, the at least one acoustic sensor is selected from the group consisting of: unidirectional microphones, bidirectional microphones, and omnidirectional microphones. In one embodiment, the at least one microphone is configured to capture a range of acoustic frequencies between about 10 Hz and 24 kHz. In one embodiment, the distance is between about 1 mm and 20 mm.

In another aspect, a electrosurgery system comprises a electrosurgery device comprising at least one ablative element, at least one microphone, and at least one power lead connected to the at least one ablative element; a power source; and a computing device; wherein the power source is electrically connected to the computing device and the at least one power lead of the electrosurgery device.

In one embodiment, the system further comprises an oscilloscope electrically connected to the power source and the at least one power lead of the electrosurgery device. In one embodiment, the system further comprises a current sensor attached to the electronic connection between the power source and the computing device. In one embodiment, the system further comprises at least one filter attached to the electronic connection between the power source and the computing device, wherein the filter is selected from a low-pass filter, a high-pass filter, and a band-pass filter.

In another aspect, a method of controlled electrosurgery comprises positioning an electrosurgery device proximate to a tissue, positioning an acoustic sensor proximate to the tissue, measuring a magnitude of an acoustic signal while applying energy to the tissue with the electrosurgery device, comparing the magnitude of the acoustic signal to a threshold, and ceasing to apply energy to the tissue with the electrosurgery device when the magnitude of the acoustic signal exceeds the threshold. In one embodiment, the method further comprises applying a conditioning filter to the acoustic signal. In one embodiment the method further comprises calculating the threshold by measuring the acoustic signal for an initial period prior to applying energy to the tissue with the electrosurgery device. In one embodiment the method further comprises calculating the mean and standard deviation of the acoustic signal during the initial period and setting the threshold to N standard deviations above the mean. In one embodiment, N is at least 6.

In one embodiment, the method further comprises collecting a window of samples of the acoustic signal of length L samples, calculating the mean, and comparing the mean to the threshold. In one embodiment, L is at least 100. In one embodiment, the method further comprises collecting a plurality of windows of samples of the magnitude of the acoustic signal and ceasing to apply energy when the mean exceeds the threshold in M consecutive windows of samples. In one embodiment, the method further comprises measuring an electrical characteristic of the energy selected from the group consisting of voltage, current, and resistance, and adjusting the energy applied based on the measured electrical characteristic and the magnitude of the acoustic signal.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1A, FIG. 1B, FIG. 1C, FIG. 1D, and FIG. 1E depict exemplary cautery devices and systems.

FIG. 2 depicts the different experimental conditions used in Experimental Example #1 presented herein.

FIG. 3 depicts a piece of porcine tissue after the bipolar tissue hemostasis process with denatures zone marked.

FIG. 4 depicts the meaning of different Sᵣ readings.

FIG. 5 depicts a graph of data collected and a curve fit for Sᵣ over time.

FIG. 6A and FIG. 6B depict plots of raw sound signal in time and time frequency domains under 35 W 50% compression level for 4 seconds. FIG. 6A depicts the magnitude of one sample sound signal plotted as a function of time. FIG. 6B depicts the spectrogram generated based on the raw signal of the same sample test.

FIG. 7 depicts bode magnitude plots of the band stop filter, raw signal after band-stop filtering only, a high-pass filter bode plot, and a raw signal after high-pass filter only.

FIG. 8 depicts plots of filtered sound signal in time and frequency domains under 35 W 50% compression level for 4 seconds.

FIG. 9A depicts the typical three-stage pattern of the sound signal generated during the bipolar tissue hemostasis process. The dashed box indicates the initial silent stage, the thinner solid boxes indicate the random popping captured, and the bold solid box shows the explosive boiling stage. The separation of different stages is based on visual inspection.

FIG. 9B depicts the filtered sound signal graph from FIG. 9A with the raw signal removed.

FIG. 10 depicts the flow chart of the detection method based on the Central Limit Theorem (CLT).

FIG. 11 depicts marked trigger points under 35 W 50% compression level for 4 seconds using a disclosed CLT based detection method.

FIG. 12 is a graph of normality rate for various window counts in different CLT based detection methods.

FIG. 13A is a graph of time delay durations and error bars for different window count thresholds in different CLT based detection methods.

FIG. 13B is a graph of false alarm rate and time delay duration for different window count thresholds in different CTL based detection methods.

FIG. 14A is a set of graphs of dynamic impedance, normalized acoustic data, and denaturated zone size over time measured under a high power setting.

FIG. 14B is a set of graphs of dynamic impedance, normalized acoustic data, and denaturated zone size over time measured under a lower power setting.

FIG. 15 depicts statistical comparison between different acoustic and impedance monitoring methods.

FIG. 16 depicts comparison of different monitoring methods under high power setting and changing displacement scenarios.

FIG. 17A and FIG. 17B depict dynamic resistance plots of high power setting with 50 W 50% for 5 seconds (FIG. 17A), and changing displacement with 35 W 50% for 4 seconds (FIG. 17B). Circled locations are potential moments that the minimal impedance monitoring method will be triggered.

FIG. 18 depicts an impedance/temperature/coagulation diagram during coagulation of a 1.5-mm diameter artery. The thermoprobe is located inside the vessel. The impedance is indicated by the dotted line and the temperature by the continuous line.

FIG. 19A depicts marks of all the triggered windows on the sound digital plot, with machine detected explosive boiling stage.

FIG. 19B depicts the marks of triggered windows and the acoustic data from FIG. 19A, with the filtered signal shown and the raw signal removed.

### DETAILED DESCRIPTION

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

The present invention provides systems, devices, and methods for electrosurgery. For example, in certain aspects, the present invention relates to dynamic monitoring of electrosurgery to prevent or reduce side effects of electrosurgery, including tissue sticking, tissue charring, thermal damage, and nerve damage. Thus, the present invention improves the quality of electrosurgical procedures while reducing the duration of the surgical procedure. In one embodiment, the present invention relates to the use of detecting and analyzing acoustic signals generated at the surgical site to provide an indication to a user on the quality of the procedure. For example, in certain aspects, the acoustic signals indicate to the user to terminate the electrical power. In certain aspects, the detection of acoustic signals automatically turn down or turn off the electrical power.

The present invention provides a reliable indicator of hemostasis formation during an electrosurgical hemostasis procedure. Due to a lack of a presently available reliable indicator, surgeons terminate the electrical power based on visual inspection. If the power is terminated too late, harmful side effects of tissue sticking and tissue charring can occur. If the power is terminated too soon, a complete hemostasis will not be formed. As described herein, the present invention makes use of a sensing element placed in the vicinity of the electrosurgical device to detect acoustic signals from the surgical site. Further, the invention comprises software and computing systems to analyze the detected acoustic signal. Analysis of the acoustic signal allows for the monitoring of the electrosurgical or hemostasis procedure and optimal termination of electrical power to reduce side effects and ensuring complete hemostasis formation.

### Electrosurgery Device and System

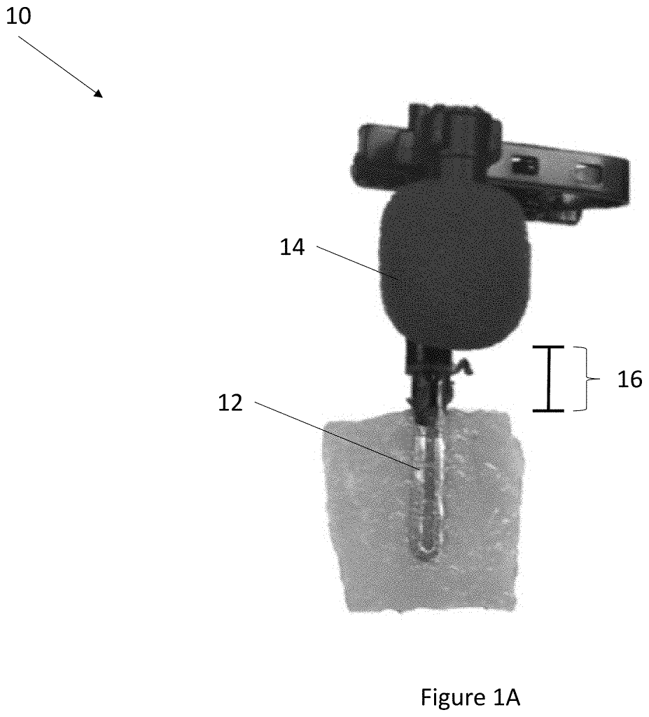

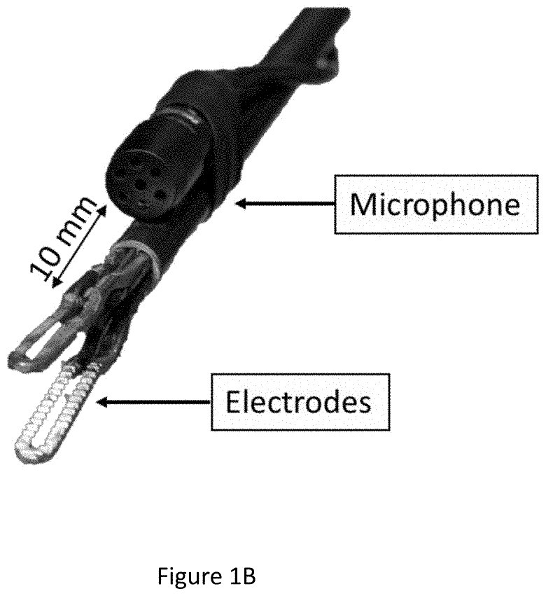

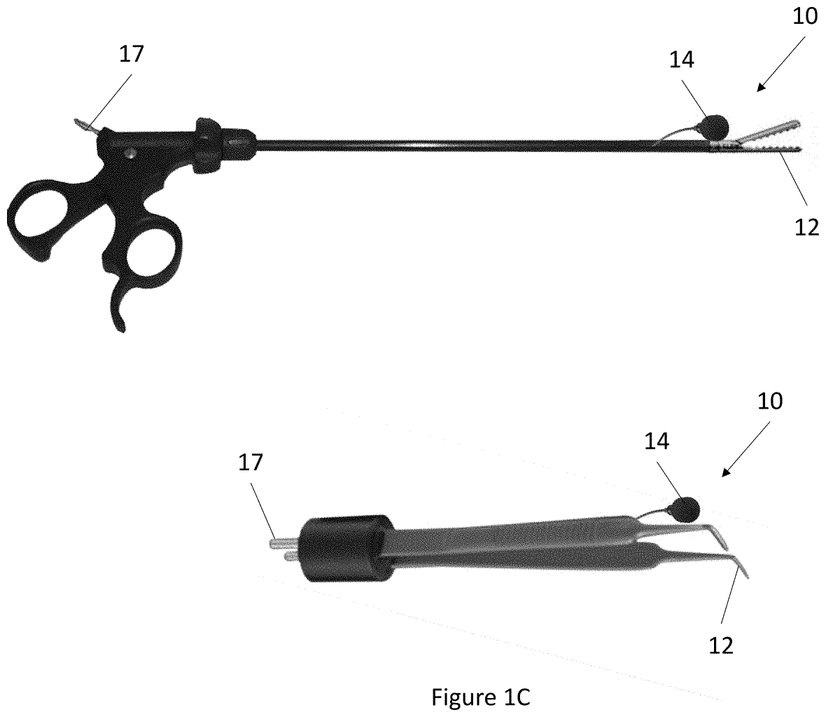

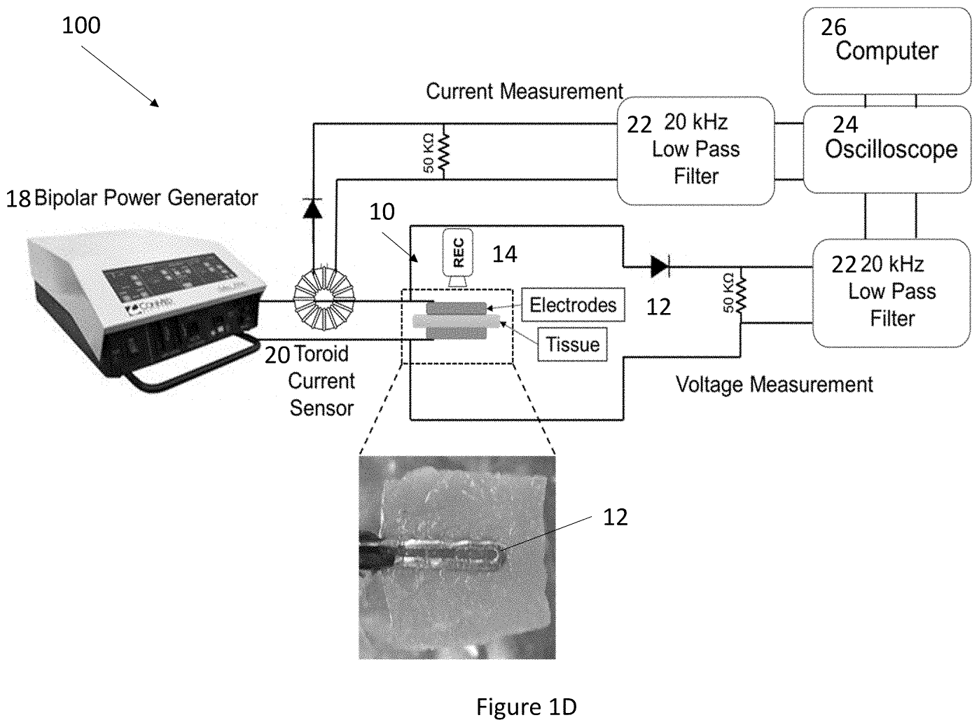

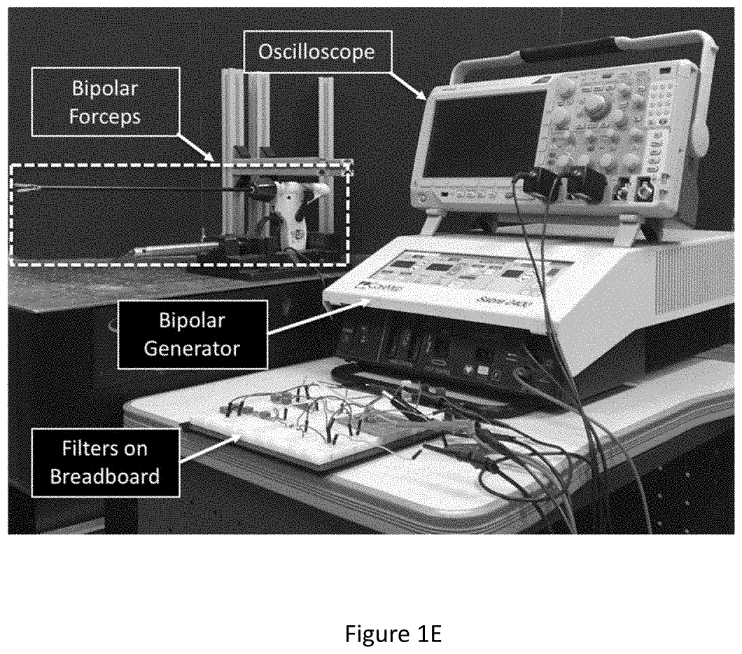

Referring now to FIG. 1A through FIG. 1E, an exemplary electrosurgery device 10 is depicted. In one embodiment, electrosurgery device 10 comprises at least one ablative element 12, at least one acoustic sensor 14, and at least one power lead 17 electrically connected to the at least one ablative element 12 (FIG. 1C). The at least one ablative element 12 can be any suitable element configurable to for temperature-based cauterization of tissue, including but not limited to bipolar electrode forceps, unipolar electrodes, laser probes, radiofrequency probes, microwave probes, and the like. Exemplary bipolar electrode forceps are depicted in FIG. 1C, including a laparoscopic forceps with a handpiece and trigger (top) and a tweezer-style forceps (bottom), each having at least one ablative element 12, at least one acoustic sensor 14, and at least one power lead 17 connectable to a power source. The at least one acoustic sensor 14 can be any suitable microphone configurable to detect acoustic signals near the at least one ablative element 12. The at least one acoustic sensor 14 can include unidirectional microphones, bidirectional microphones, and omnidirectional microphones. The at least one acoustic sensor 14 is configured to capture a range of acoustic frequencies, such as in the range of between about 8 and 88 kHz. In some embodiments, the at least one acoustic sensor 14 can include a windscreen or pop filter. The at least one acoustic sensor 14 can be positioned at a distance 16 from the at least one ablative element 12. Distance 16 can be any suitable distance, such as between about 1 mm and 20 mm.

Referring now to FIG. 1D, electrosurgery device 10 is depicted being electronically connected to electrosurgery system 100. Electrosurgery system 100 comprises power source 18, current sensor 20, oscilloscope 24, and computing device 26. Power source 18 can be any suitable source of power configured to supply an amount of energy to power leads 17 of electrosurgery device 10, such as a bipolar or unipolar power generator. Current sensor 20 can include but is not limited to Hall effect sensors, integrated circuit sensors, fiber optic sensors, Rogowski coil/toroid sensors, and the like. Electrosurgery system 100 is configured to measure current using current sensor 20, oscilloscope 24, and computing device 26, and can measure voltage using oscilloscope 24 and computing device 26. In some embodiments, electrosurgery system 100 can include one or more filters 22 for filtering a measured signal. The one or more filters 22 can include low-pass filters, high-pass filters, band-pass filters, and combinations thereof.

In some embodiments, electrosurgery system 100 can be provided with at least one acoustic sensor 14. The combination of electrosurgery system 100 with at least one acoustic sensor 14 can be combined with an existing electrosurgery device to add acoustic signal receiving and interpreting capabilities. The at least one acoustic sensor 14 can further include a clip, adhesive, clamp, or other mechanism to facilitate attachment to an electrosurgery device. Suitable electrosurgery devices include bipolar electrode forceps, unipolar electrodes, laser probes, radiofrequency probes, microwave probes, and the like.

The present invention also provides software for controlling electrosurgery device 10 and electrosurgery system 100. The software is configured to manage the amount of power supplied from power source 18 to electrosurgery device 10 to achieve a desired temperature at the at least one ablative element 12. The software is also configured to interpret acoustic signals captured by the at least one acoustic sensor 14 and to modulate the amount of power suppled from power source 18 based on the interpreted acoustic signals.

### Controlled Electrosurgical Method

In one embodiment, a method of the present invention comprises one or more data collection and analysis steps for determining when to terminate the delivery of thermal energy to tissue during an electrosurgical or hemostasis procedure. Steps of the method are performed by a signal processing and control system electrically connected to one or more electrosurgery devices configured to deliver thermal energy to tissue, for example during an electrosurgery or hemostasis process.

The present method can be used to monitor and control various electrosurgical procedures, including but not limited to electrocautery, hemostasis, or any other procedure where electric current is used to cut, coagulate, desiccate, or fulgurate tissue. In one embodiment, the method provides the monitoring of an electrosurgical hemostasis procedure to seal a blood vessel or to otherwise stop or prevent bleeding. The present method may be used during electrosurgical procedures in any suitable tissue including, but not limited to, brain, skin, muscle, heart, cardiac tissue, tonsils, the spine, and the like. In one embodiment, the method is used in electrosurgical procedures performed on the extremities, including, but not limited to arms, legs, hands, feet, fingers, and toes.

Existing machine-assisted monitoring methods include impedance monitoring, temperature sensing and temperature mapping. These methods improve the quality of bipolar tissue hemostasis over simple visual inspection. However, these monitoring methods cannot guarantee the outcome of joint quality. The minimal impedance monitoring method has been used by certain equipment vendors as an indicator of when it is appropriate to stop delivery of heat energy. However, the impedance measurement can be affected by many factors, including but not limited to welding site irrigation, changing displacement between electrodes, and having a high power setting during the bipolar tissue hemostasis process.

The initial impedance measurement between the electrodes has also been used to determine the amount of energy to be delivered to the tissue. However, such pre-calibrated generators are extremely sensitive to tissue compression, damaged electrode coating, and or residual tissue sticking conditions, which will change the impedance measurement conditions thus leading to incorrect feedback signals. These in turn strongly affect the initial impedance measurement, which skews the effectiveness of further impedance measurements in the same tissue. As a result, in current electrosurgical operations, side effects such as tissue sticking, charring, and excessive thermal damage often occur, sometimes leading to fatal complications under extreme circumstances.

A controlled electrosurgical method of the present invention is divided into setup steps, measurement and recording steps, heat application steps, processing steps, and controlling steps. Methods of the present invention may exercise closed loop or open loop control, though closed loop control is advantageous for the purposes of the invention. Referring to FIGS. 1A and 1C, in a setup phase of the present invention, an electrosurgical device 10 is placed such that one or more ablative elements or electrodes 12 are in contact with tissue, the electrosurgical elements further electrically connected to power source 18. The power source 18 may be configured to deliver any suitable power setting to the ablative elements 12, including but not limited to 25 W, 35 W, 45 W, or 50 W. In some embodiments, the power delivered may vary across different treatments or during a single treatment, and may be dynamically set by a clinician or operator. Suitable voltages of systems of the present invention may include for example between 10V and 120V rms.

A microphone or other acoustic sensor 14 is placed a distance 16 from the tissue. In some embodiments the distance 16 is fixed regardless of the tissue or other parameters of the procedure, while in other embodiments the distance 16 may be varied depending on the tissue type or a variety of other parameters, including but not limited to air temperature, humidity, electrosurgery device size, or the nature or settings of one or more recording or data collection devices elsewhere in the control system. In one embodiment, the distance 16 is about 10 mm, but suitable distances include the range from 1 mm to 50 mm, 5 mm to 20 mm, or any other suitable distance. The microphone or other acoustic sensor 14 may be fixedly attached to a bipolar forceps, or may alternatively be held in position by other means. The microphone or other acoustic sensor 14 may further comprise a pop filter or other muffling element in order to reduce undesirable noise and or clipping effects.

A setup phase may further include a compression step, wherein electrodes 12 are placed on the tissue with a pressure determined by the “compression level,” which is a measurement of the decrease in thickness of the tissue caused by the application of the electrodes. For example, if the electrodes are incorporated into a robo-surgical forceps and the tissue at rest was 10 cm thick, squeezing the electrodes into place on the tissue with a force sufficient to compress the tissue to 5 cm thick would yield a compression level of 50%. Suitable compression levels of the present invention include, but are not limited to, 0%, 10%, 25%, 50%, 75% or any other suitable ratio. In some embodiments, a method of the present invention may vary the power applied based on the compression level, but in other embodiments a method of the present invention may use a fixed power applied and compression level.

Measurement steps of a process of the present invention may include acquisition of acoustic signals from an acoustic sensor 14, for example sound pressure measurements at a sampling rate. Suitable sampling rates include, but are not limited to about 44.1 kHz, about 48 kHz, about 96 kHz, about 250 kHz, or any other suitable sampling rate. It is understood that where a digital measurement step of the present invention requires measurements of analog data up to a given maximum frequency, the (Nyquist) sampling frequency must be at least twice the maximum measured frequency. In some embodiments, a method of the present invention further includes sampling and recording of current and/or voltage values measured from the power supply in order to generate a calculated power measurement. A current sensor of the present invention may include a toroid current sensor, a Hall effect current sensor, an “amp clamp,” a small-value resistor based current sensor, or any other suitable device for measuring current. Prior to digital sampling, some or all of the analog signals of a method of the invention (including but not limited to the sound pressure waveform, the voltage waveform, and the current waveform) are run through a signal conditioning filter. In some embodiments, the signal conditioning filter may be a low-pass filter, a high-pass filter, or a band-pass filter. Filters used in methods of the present invention may be active or passive. In some embodiments, the signal conditioning filter is a combination of one or more of these. In one embodiment, the signal conditioning filter comprises a Butterworth filter. In the exemplary embodiment depicted in FIG. 1A, the voltage measurement and the current measurement are each passed through a 20 kHz low pass filter 22.

A data acquisition step of the present invention may be performed by a data acquisition device 24, for example an oscilloscope, electrically connected to one or more of the analog signals. In some embodiments, the one or more analog signals are conditioned first by a signal conditioning filter, while in other embodiments, some or all of the signal conditioning is performed after the signal is converted to a digital waveform by sampling. The data acquisition device of the present invention may comprise one or more analog-to-digital converters (ADCs) which perform sampling and quantization of one or more analog signals to convert the signals to digital values. Once converted, the one or more recorded signals of the present invention may be analyzed by a software module on a computer 26 in order to determine when to terminate the connection between the power supply 18 and the electrodes 12.

In some embodiments, the measurement and recording phase of a method of the present invention may be divided into an initial measurement window and an active measurement portion. The initial measurement window may be performed at the beginning of the analysis, before any heating has taken place in the tissue. In one embodiment, the initial measurement lasts 0.25 seconds, while in other embodiments it may be longer or shorter depending on the application. The initial measurement period may be used to gather baseline data, to which the active measurement data can be compared. During the initial measurement window, a limited number of data samples are gathered, in some embodiments using a windowing function whereby a set of thousands of samples is divided into multi-sample “bins,” and each bin's mean value is calculated. The means of each bin are then averaged together to yield an overall mean and standard deviation (and probability density curve) of the initial measurement window data. The overall probability density curve serves to characterize random background noise in the system, and the standard deviation can be used to calculate a threshold above which the system can determine with statistical certainty that a future measured signal is outside the background noise range. In some embodiments, the threshold is set at 6 standard deviations above the mean, but in other embodiments 2 standard deviations, 3 standard deviations, or other different thresholds may be used as appropriate.

During an active measurement step, the measured signal is grouped into windows and averaged, with the average value compared to the threshold calculated during the initial measurement period. In some embodiments, the measured signal may be rectified during the initial and/or active measurement periods, for example half-wave rectified, for example with a diode, or may alternatively be full-wave rectified, for example with a bridge rectifier. When the average value of a window exceeds the calculated threshold, that window can be considered “triggered” for the purposes of measuring the signal of interest. In some embodiments, transient triggering events (sometimes called ‘pops’) are detected, and so a further windowing or de-bouncing algorithm is applied to the triggered windows. The de-bouncing algorithm has the effect of filtering out transient triggering events by waiting for n windows to be triggered consecutively before determining the legitimacy of the triggering event. In this way, a transient pop, which may only trigger one or two windows, will not result in a positive measurement, while a sustained noise will trigger the required number of consecutive windows and indicate a positive measurement. The value of n may in some embodiments be 5, 10, 20 or more depending on the application.

Once the de-bounced signal indicates a positive measurement, a method of the present invention may implement one or more control steps, feeding back into the system. In one embodiment, the control steps comprise shutting down the power supply, and in some embodiments the control steps comprise releasing a set of robotic forceps, activating a visual and/or acoustic alarm, cutting off power to all or part of the system, or another suitable control step.

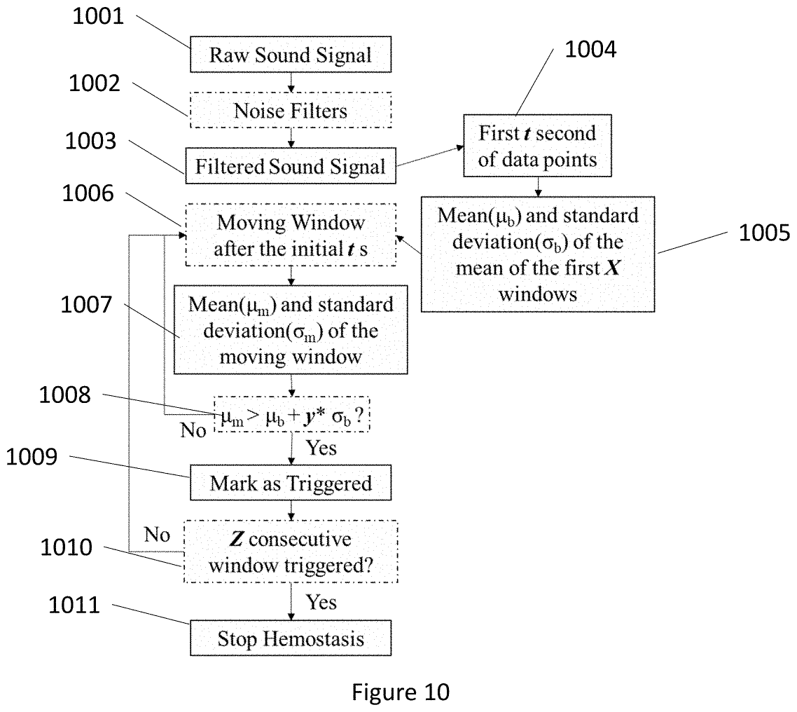

An exemplary control method is shown as a hybrid system diagram and decision tree in FIG. 10. The raw sound signal is first sampled 1001 and subjected to one or more noise filters 1002. The filtered sound signal 1003 is then first used to calculate the initial data set, comprising in this exemplary embodiment the first 0.25 seconds of data points 1004. The mean and standard deviation (and probability density curve) of the initial data set is calculated 1005, along with the accompanying threshold, after which the active measurement phase begins. In the active measurement phase, a moving window of audio data is sampled 1006 and a mean of the moving window is calculated 1007. If the mean of the moving window is below the threshold established by the initial data set, the trigger counter is reset to zero and the next window is sampled. If the mean of the moving window is above the threshold established by the initial data set 1008, the trigger counter is incremented 1009, and the next window is sampled 1006. Once the trigger counter reaches the predetermined threshold n, which in the depicted example is 10 consecutive windows 1010, the control step is performed 1011, which in the depicted example is shutting down the hemostasis process.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art may, using the preceding description and the following illustrative examples, utilize the present invention and practice the claimed methods. The following working examples therefore, specifically point out exemplary embodiments of the present invention, and are not to be construed as limiting in any way the remainder of the disclosure.

In the below examples, a novel acoustic signal monitoring method was demonstrated for improving the quality of electrosurgical procedures. A microphone was used to collect the sound signal during the bipolar heating process. Experiments with porcine muscle were conducted under various process conditions, including different heating power, time, compression force, and displacement. An algorithm based on the central limit theorem was developed to monitor the hemostasis process. The result was compared with those using the minimum impedance monitoring method.

A novel sound signal monitoring method is developed in this study for bipolar tissue hemostasis. Various process conditions are tested to validate the performance of the new method. Using a simple detection algorithm, the sound signal monitoring method is compared with the well-known minimal impedance monitoring method. The results show significantly improved performance by the sound signal monitoring. The advantages of using the sound signal monitoring method include robustness against variation of process settings and conditions, consistent hemostasis outcome, higher success rate and low false alarm rate, reduction in sticking and surgical smoke, and the ability to detect accurately the completion of the hemostasis process, leading to more confidence in bipolar electrosurgery operations.

### Experimental Setup

FIG. 1D shows a schematic of the experimental setup. A bipolar electrosurgical generator was used to generate the power needed for the hemostasis process. A commercial minimal-invasive hand-piece was used to deliver the heating energy to the tissue samples. The heating current and voltage signal was recorded using an oscilloscope via a customized toroid sensor and lead wires respectively. These two signals were used to calculate the dynamic bio-impedance of tissue during the hemostasis process. Two analogue low pass filters were used to help reducing the required sampling frequency. A sampling rate of 250 kHz was used to sample the 20 kHz pulse train from the generator, which conveys enough information for impedance estimation. FIG. 1A shows a close-up photo of one exemplary microphone setup, while FIG. 1B shows an annotated close-up photo of another exemplary microphone setup. Each test was recorded using a video camera. The sound signal was recorded with an external lavalier microphone connected to the camera. The microphone was placed 10 mm from the electrodes with the diaphragm directed towards the tissue. An annotated photograph of the experimental setup is shown in FIG. 1E.

### Experimental Procedure—Experiment #1

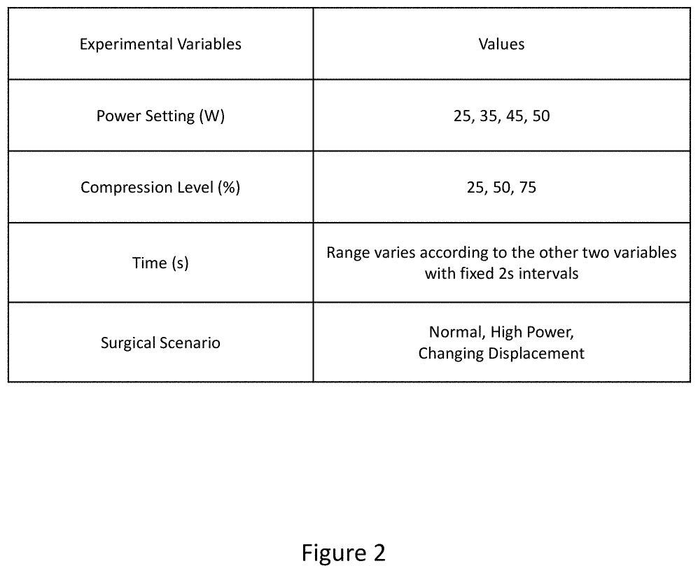

Excised porcine muscle samples with dimensions of 20×20×5 mm were thawed to room temperature before each test. Excess water on the tissue surface was removed with absorbent wipes before it would be placed between the two electrodes. Tests were performed with various power, duration, compression level, and surgical scenario settings. FIG. 2 shows the different experimental conditions used. The compression level is the ratio of the thickness decrease of the compressed tissue over the initial tissue thickness. For fixed compression level tests, the locking mechanism of the hand piece was used during the tests. For changing displacement tests, 35 W power and 50% compression ratio were selected. The tissue sample was compressed to 50% compression level initially by pulling the handle by hand. Then the same operator would push and pull the handle slightly to mimic the change of displacement between the two electrodes during real operations. High power is used for circumstances where the surgeon needs to denature the target tissue within a short duration. Under high power settings, 50 W and 50% compression level were selected. Each of these two scenarios has 5 different welding times tested.

The video recording started before the power of the bipolar generator was turned on for each test. The exact starting and ending point of each test were determined during post processing, when the sound signal was extracted from the video files. The sampling rate of the sound signal was set to be 48 kHz. The audio files were later imported into MATLAB for further analysis. Top surface image of each heated sample was taken after each test to examine the size of the denatured tissue zone. The electrodes were cooled down and cleaned after each test.

### Experimental Procedure—Experiment #2

Excised porcine muscle samples with dimensions of 20×20×5 mm were thawed to room temperature before each test. Excess water on the tissue surface was removed with absorbent wipes before the sample was placed between the two electrodes. Tests were performed with various power, duration, compression level, and surgical scenario settings. Table 1 below shows the experimental conditions used. The compression level is the ratio of the thickness decrease of the compressed tissue over the initial sample thickness. For the changing displacement tests, 35 W power and 50% compression ratio were selected. The tissue sample was compressed 50% initially by squeezing the handle manually. Then the same operator slightly released and squeezed more to mimic the unsteady clamping of the tissue during real operations. A high power setting is used for circumstances where the surgeon needs to denature the target tissue within a short duration. Under high power settings, 50 W and 50% compression level were selected. Each of these two scenarios was tested for 5 times. The sampling rate of the sound signal was set to be 48 kHz. The top view of each sample was taken after the test to examine the size of the denatured tissue zone. The electrodes were cooled down to room temperature and cleaned after each weld.

TABLE 1

| Experimental Variables | Values |
|---|---|
| Initial Compression Level | 50 |
| (%) | |
| Power Setting (W) | 35, 50 |
| Welding Time (s) | Range varies according to the |
| | other two variables with |
| | fixed 2 s intervals |
| Surgical Scenario | High Power |
| | Changing Displacement |

### Response Variables

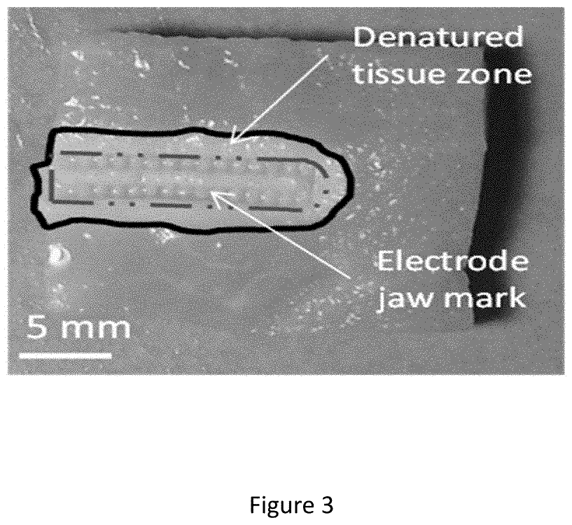

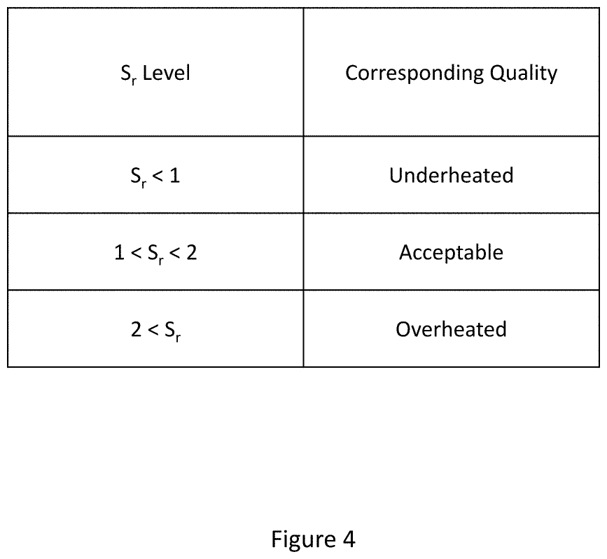

The change in color of the target tissue is the first and the most important sign for surgeons to decide whether hemostasis is formed during surgeries. In this study, a relative denatured zone size (Sᵣ) is introduced and used as the indicator of the quality measure. FIG. 3 shows a photo of a porcine sample after the hemostasis procedure. The outer solid line shows the denatured surface area, whereas the dotted line shows the mark of the electrode. The Sᵣ is calculated as the ratio of the total denatured tissue zone to the size of the electrode, which was roughly 55 mm² in this study. The exact size of the denatured tissue zone for each sample was measured using the image processing software, ImageJ, with the help of visual inspection. FIG. 4 depicts a table that defines the meaning of different Sᵣ reading. When Sᵣ<1, not all the tissue underneath the electrodes is denatured, which is defined as underheated. When 1<Sᵣ<2, all the tissue underneath the electrodes is denatured, while the denatured zone is still not widely spread. The Sᵣ reading in FIG. 2 is around 2. With Sᵣ reading larger than 2, the lateral thermal damage is overwhelming, and the tissue is considered to be overheated. There is also a high risk of sticking and charring.

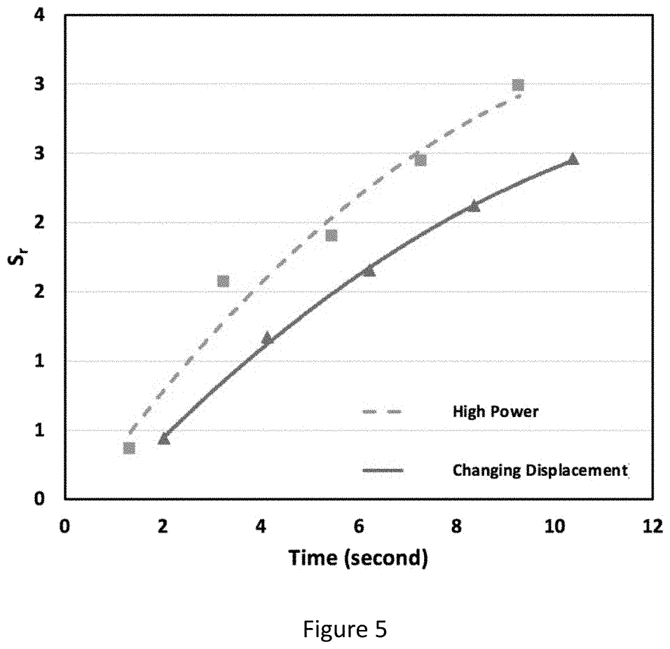

The relationship between the heating duration and the expected Sᵣ is generated based on regression. FIG. 5 shows measured values and corresponding fitted curves for the estimated expressions of Sᵣ as a function of heating duration for high power and changing displacement settings. These functions are used later to determine Sᵣ at the moment when the power is stopped using different monitoring methods.

### Sound Signal Conditioning

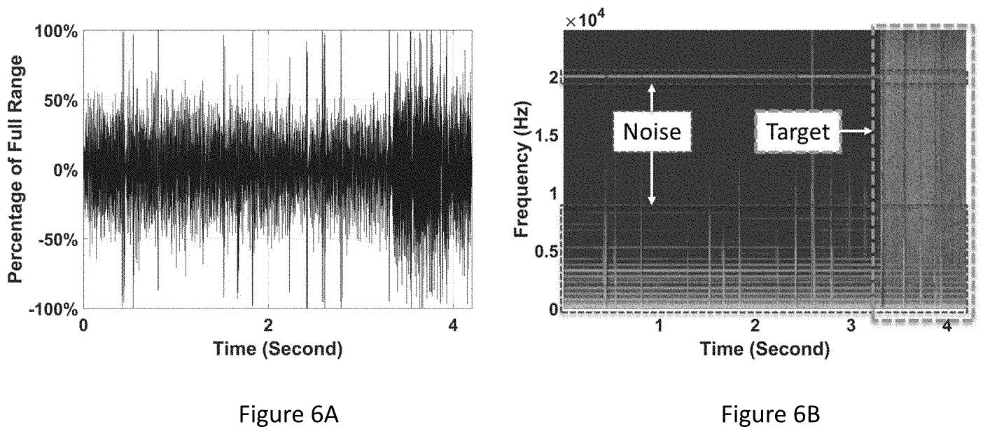

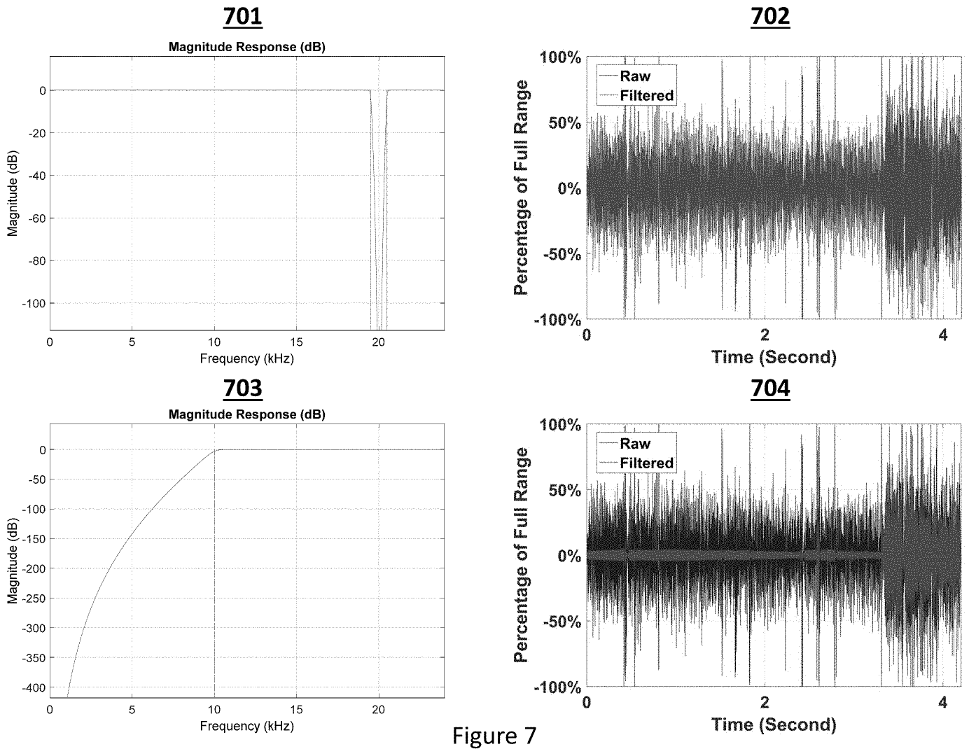

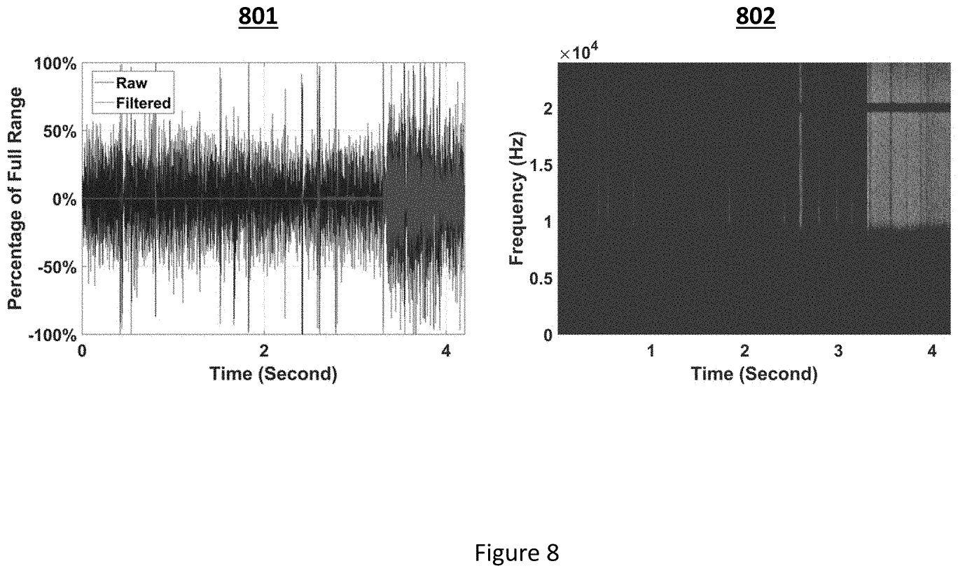

The imported raw sound signals were examined in both the time and frequency domains. FIG. 6A shows the magnitude of one sample sound signal plotted as a function of time. This reveals the loudness of the sound signal as time elapses. As for the frequency domain analysis, the raw signals were analyzed using a Short-Time Fourier transform (STFT) using MATLAB. A window size of 512 samples, which is roughly equivalent to 10 milliseconds in the time domain, and an overlap of 256 samples were used in the STFT analysis. FIG. 6B shows the spectrogram generated based on the raw signal of the same sample test. In general, the frequency components in the raw signal can be divided into three categories: the consistent background noise at 20 kHz, random popping sounds, and the target sizzling sound signal generated due to the explosive boiling of tissue water content (Ward A K et al., Engineering in Medicine and Biology Society, 2007, 1180-1183). The background noise needed to be removed before the signal could be further analyzed. Filters were designed to remove the noise components based on the spectrogram analysis. A 20^{th} order digital Butterworth notch filter with cutoff frequencies of 19.5 and 20.5 kHz was used to remove the 20 kHz noise, while a 20^{th} order Butterworth high-pass filter with a cutoff frequency of 10 kHz was applied to remove low frequency noise components. Graphs 701 and 703 in FIG. 7 show the bode plot of the notch and the high-pass filter, respectively. While graphs 702 and 704 show the plot of the signal after the notch and the high-pass filter, respectively. FIG. 8 shows the signal after the two filters in both time (801) and frequency domain (802). The depicted sound signal was measured with the instrument operating at 35 W, and with an initial compression level of 50%.

### The Understanding of Sound Signal During Bipolar Tissue Hemostasis Process

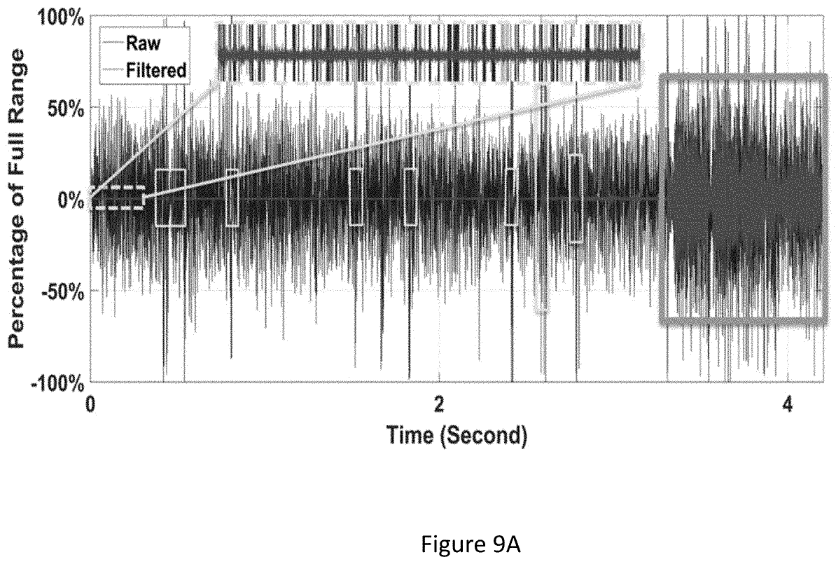

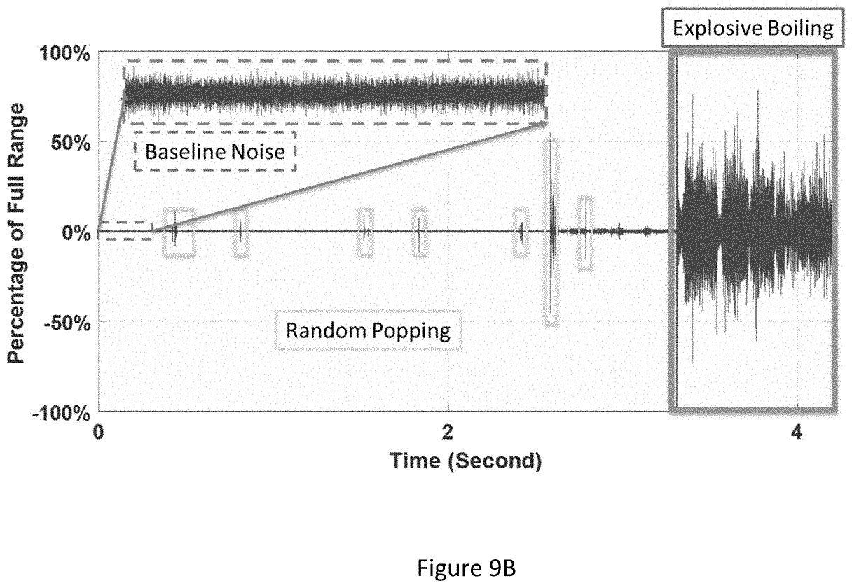

Graph 801 in FIG. 8 reveals that the general sound signal captured during the bipolar tissue hemostasis process consists of the background noise, random popping sound, and the sizzling sound. Ward et al. defined the electrosurgical tissue heating process as two different stages, which are the confined and explosive boiling stage (Ward A K et al., Engineering in Medicine and Biology Society, 2007, 1180-1183). The confined water boiling stage is before the pressure buildup exceeds the fracture strength of the tissue, so that no acoustic signal other than random popping sounds could be captured. Once the pressure buildup inside the tissue due to water vaporization exceeds the tissue fracture strength, steam escapes from the tissue and generates a detectable sizzling and bubbling sound. Based on the observations of filtered sound signals from the 91 tests conducted, a typical three-stage pattern of the sound signal generated during the bipolar tissue hemostasis process is established in this disclosure. FIG. 9A shows the three stages based on visual inspection, which are the initial silent stage, confined boiling popping stage, and explosive boiling stage. FIG. 9B shows the same graph with the raw audio signal removed for clarity. The hemostasis process entered the explosive boiling stage when the inner temperature reached the boiling temperature of the water content. Thus, the moment that the explosive boiling stage starts reflects the a certain degree of completion of the welding process. It was hypothesized that the optimal moment to stop the hemostasis process is the start of the explosive boiling stage. Therefore, a change detection method is applied to the acoustic signal to identify the start of the explosive boiling stage in each test. The corresponding denatured zone size (Sᵣ) is used to represent the welding quality and to validate the hypothesis.

### The Change Detection Method for Sound Signal Analysis

A detection method based on the Central Limit Theorem (CLT), which is the foundation of the Shewhart Chart, was developed to determine the start of the explosive boiling stage. FIG. 10 shows a block diagram of the algorithm. Instead of using the raw data, the signal is first rectified so that all the values are positive. For each test, the first t seconds of the sound signal is evenly divided into x windows, with n points in each window. According to CLT, the means of the X windows, when X is large, tend to follow a normal distribution regardless of the original distribution. With the mean and the standard deviation of the X window-means denoted as μₕ and σₕ, respectively, the probability density function (PDF) of the window-mean can be expressed as:

$$f ( x ) = \frac{1}{\sqrt{2 π {σ}_{b}}} e \frac{{( x - {μ}_{b} )}^{2}}{2 {σ}_{b}^{2}} \qquad Equation 1$$

A moving window with the same n data point window size was then applied to the sound signal after the initial t seconds, assuming the hemostasis would not form within the first t seconds. The sample mean of the moving window, μₘ, was monitored. The window was marked as triggered if the following relationship held true:

μₘ≥μ_{b}+y·σ_{b} Equation 2

where y is a constant determining the confidence level that the mean of the moving window is not from the same distribution as the initial base data. In one example, when y is 6, the confidence level is a six-sigma criterion, which represents a 99.999999% confidence level that the mean of the moving window is not from the same distribution as the initial base data, i.e.,

$$P ( {μ}_{m} > {μ}_{b} + 6 {σ}_{b} ) = {\int }_{6 {σ}_{b}}^{∞} \frac{1}{\sqrt{2 π} {σ}_{b}} {e}^{- \frac{{( x - {μ}_{b} )}^{2}}{2 {σ}_{b}^{2}}} = 0.0000000987 \% \qquad Equation 3$$

When Z consecutive windows are triggered, a decision is made to stop the hemostasis process.

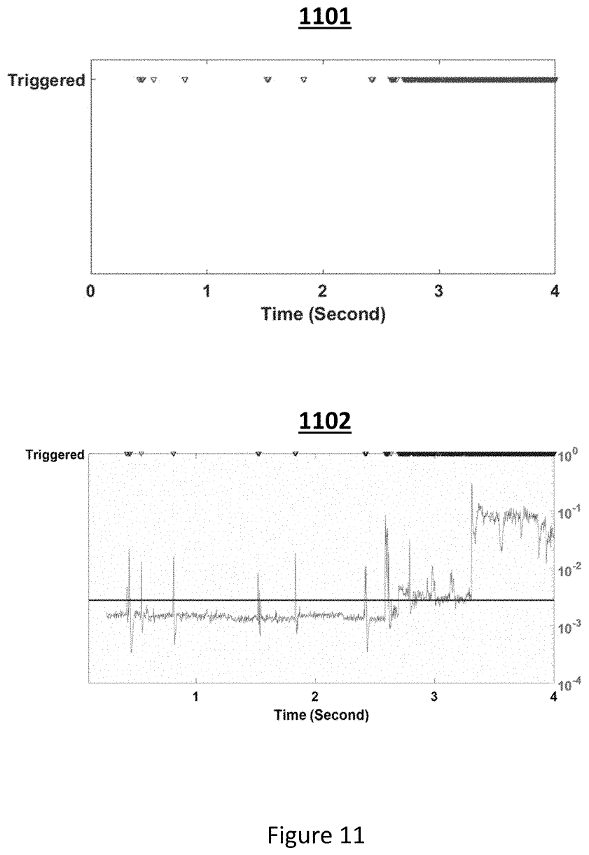

FIG. 11 shows the locations of all triggered windows of one sample with 35 Watts, 50% initial compression level, and changing displacement setting. Graph 1101 shoes the triggered window locations only, while graph 1102 shows the triggered window locations along with the μₘ and the corresponding threshold. For the depicted example, t=0.25 seconds, x=100 windows, n=120 points per window, and y=6, leading to a 99.999999% confidence level that the mean of the moving window is not from the same distribution as the initial baseline data, as discussed above. The moving window can be triggered under two circumstances, which are the capture of popping sound and the explosive sizzling sound. The duration of the popping sound captured before the explosive boiling stage is short in nature. Thus, an anti-false-alarm mechanism was introduced in this CLT based detection method. The indicator of hemostasis completion would be turned on only if Z consecutive moving windows, were triggered during the monitoring process.

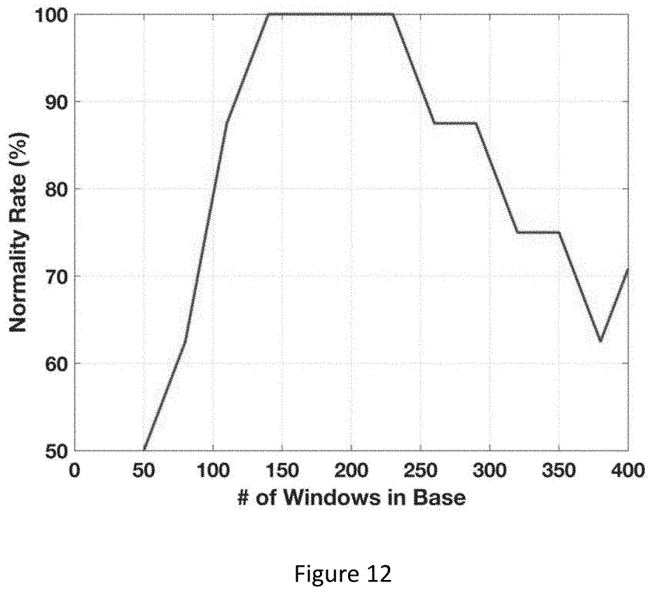

The parameters to be determined in the decision rule included the initial time period t used as the baseline data, the number of windows X to establish the window mean, the constant y for determining the control limit, and the number of consecutively triggered windows Z to make a final decision. Based on observations, t was selected to be 0.25 seconds to avoid incorporating any popping sound signal into the baseline data. This initial period of sound signal contains 12,000 discrete data points with a 48 kHz sampling frequency. The rule-of-thumb to satisfy the Central Limit Theorem is to have a sample size larger than 30. At the same time, there should be at least 30 data points in each window. To select an appropriate number of windows (X), the Shapiro-Wilk Normality test was conducted with the number of windows varying from 30 to 400. Correspondingly, the number of data points in each window (n) varied from 400 to 30. FIG. 12 shows the normality test results, with a 100% normality rate indicating a normal distribution of the window mean. Based on the depicted results, X was chosen to be 100 and n was chosen to be 120 in the disclosed example. The selection of y in the control limit determines the confidence level of the decision-making process. As the baseline data has a signal strength several orders of magnitude lower than the popping and explosive boiling sound, y was set to be 6 for a high confidence level, as discussed above.

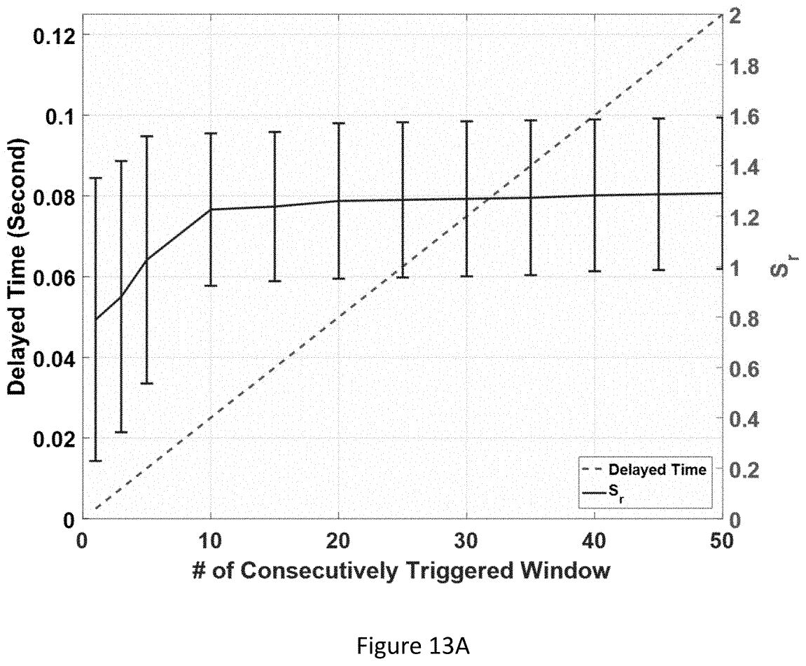

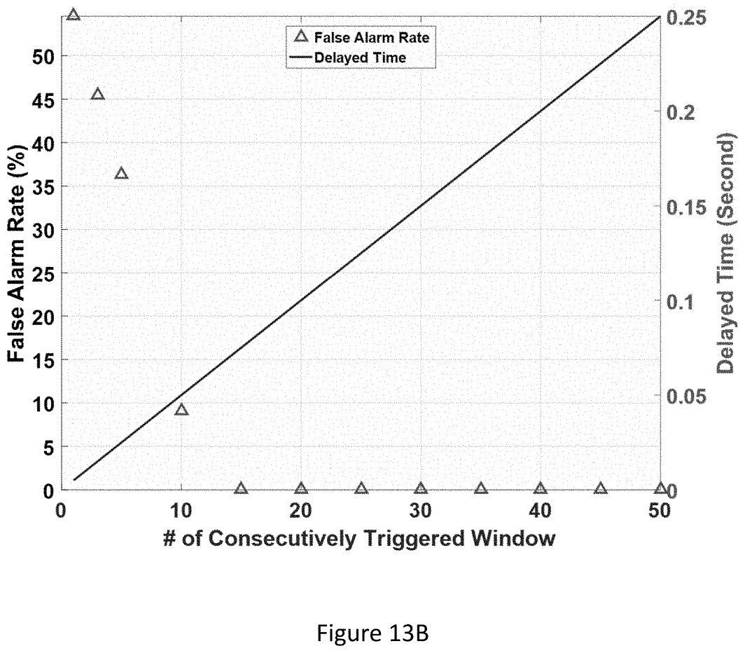

Another important parameter in the monitoring algorithm is the number of consecutively triggered windows that will lead to the decision of terminating the hemostasis process. FIG. 13A plots the Sᵣ and delayed time under a different number of consecutively triggered windows used to make the decision of stopping the electrical power. The Sᵣ reading when Z is less than 10 all have an error bar greater than one sigma, which indicates larger variation compared to a Z larger than 10. When Z was set to 1 and 3, the corresponding Sᵣ are less than 1, which is due to triggering caused by popping. As shown in the graph of FIG. 13A, results are comparable for increasing values of Z beyond 10, but with an increased time delay. The difference between using Z=10 and Z=50 are 0.06 in Sᵣ reading and 0.1 s in time delay. FIG. 13B shows the false alarm rate plot with delayed time as a function of Z. The false alarm rate is defined as the probability that the monitoring method would be triggered 0.5 seconds prior to the explosive boiling stage, as measured visually. With all the tests conducted, the false alarm rate is 0 for all values of Z greater than 10. Based on the results in FIG. 13B, Z values of Z greater than or equal to 15 should be used to achieve optimal results, while a larger Z leads to increased time delay to stop the hemostasis process. Z was set to 20 in this study due to a safety factor. The parameters detailed above were used to generate projected monitoring results to be compared with the minimal impedance monitoring method.

Comparison with Traditional Dynamic Impedance-Based Monitoring

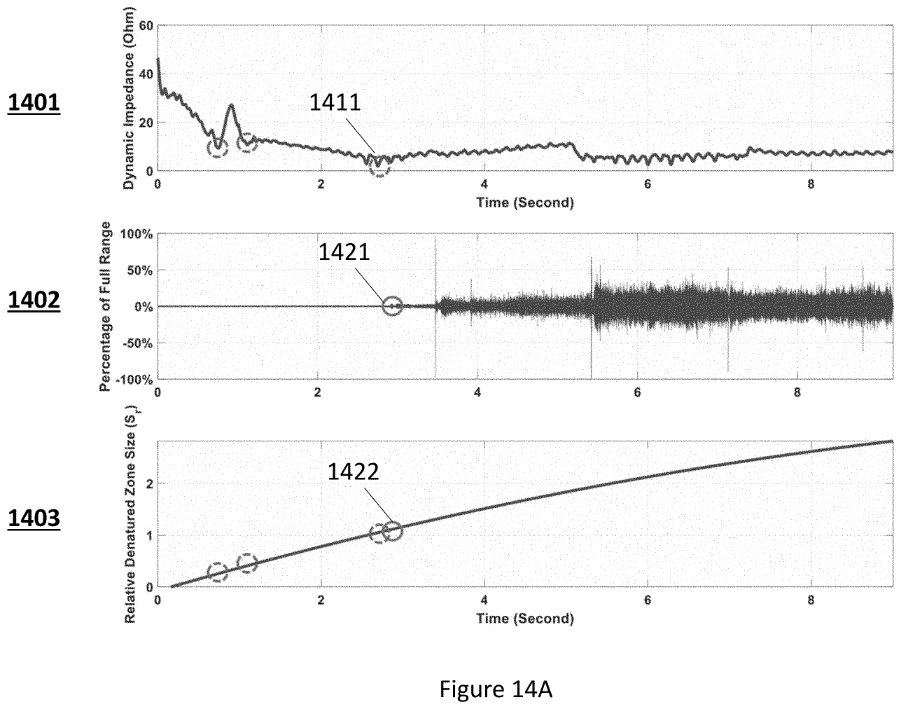

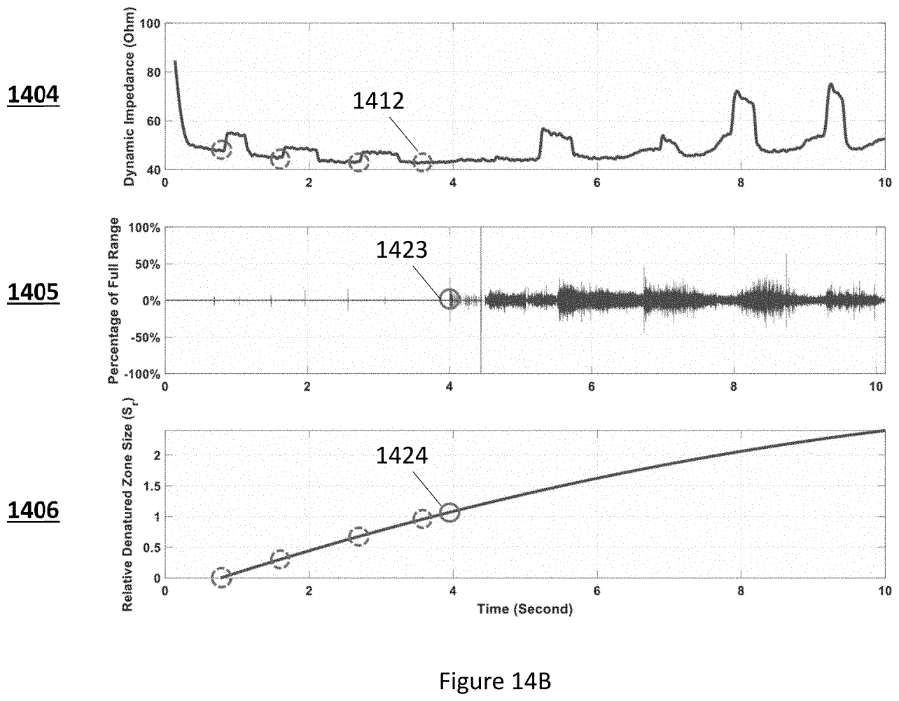

FIG. 14A and FIG. 14B show the typical dynamic resistance plots for the high-power setting and the changing displacement scenarios with the corresponding sound signal and duration-Sᵣ plot. The high-power setting made the water in tissue heat faster, causing the temperature of some parts in the tissue reaching the boiling point faster than others. The vaporization of local water content leads to increased impedance. The minimal impedance monitoring method triggered before the actual completion of the hemostasis process due to local vaporization. The same mechanism was in play for the changing displacement scenario, where the displacement between the two electrodes was not kept at the same level throughout the hemostasis process. When the contact area changes, the impedance reading also changes even if the tissue has not been fully denatured.

These factors, however, did not influence the performance of the sound signal monitoring method due to the nature of sound generation regardless of the power settings and compression conditions applied. The sound signal monitoring method detected the sound signal generated when the temperature inside the tissue reached the same level. FIG. 14A and FIG. 14B reveal that there were multiple local impedance minimums (see graphs 1401 and 1404) before the global minimums (1411 and 1412) were measured. The dashed circles indicate all the local minimums including the global minimum as the last one for impedance signals, while the solid circles (1421 and 1423) show the detected start of the explosive boiling stage for sound signals. The corresponding Sᵣ readings are marked on the duration-Sᵣ plot (1422 and 1424). Depending on the design of the algorithm, the local minimum impedance measurements could potentially trigger the monitoring method to stop the hemostasis process at an inappropriate time. In one embodiment of the method disclosed herein, if a minimum impedance was detected and remained the minimal with 0.2 second delay then the monitoring method stops the hemostasis process. The graphs of FIG. 14A show data measured using a high power setting of 50 W with a 50% initial compression level over 9 seconds. The graphs of FIG. 14B show data measured using a lower power setting of 35 W, with 50% initial compression level over 10 seconds. The circled locations are potential moments that a minimal impedance monitoring method might be triggered.

The results of the experiments are now described.

### Results—Experiment #1

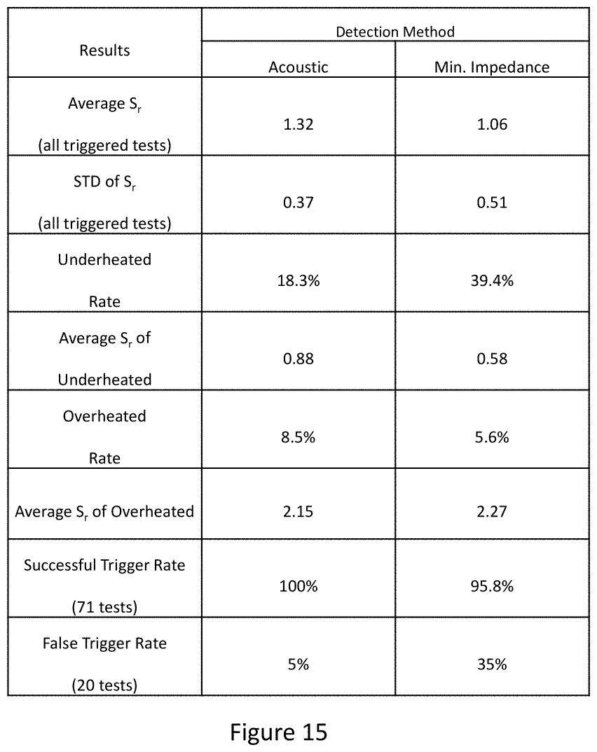

FIG. 15 shows the comparison of results using different monitoring methods for 91 tests. After manual check of all the tests, 20 of them are too short to complete the hemostasis process, while an effective detection method should be able to identify the start of explosive boiling stage for the other 71 tests. Overall all, the minimal impedance monitoring method resulted in a large standard deviation on the denatured tissue zone, which means that more likely a result would fall outside the acceptable range. The impedance monitoring method yielded 39.4% of triggering while the tissue was underheated, while the CLT based monitoring method had an only 18.3% underheated rate. Within all the underheated cases, it was found that on average 58% of the tissue underneath the electrodes was denatured with the minimal impedance monitoring method. The 1.06 overall average Sᵣ is not as impressive as it seemed, because the 40% underheated cases were with an average of 0.58 for Sᵣ. On the other hand, around 88% of the tissue was denatured when the CLT based monitoring method was triggered before the desired result was achieved. For overheated cases, all the monitoring methods have comparable results. For all the 71 tests that should have been detected, both methods performed similarly, with the minimal impedance method had the lower 95.8% success rate. As for the 20 tests that should not trigger any detection method, the CLT based method had a 5% of false trigger rate, while the minimal impedance method had a 35% false trigger rate.

These comparisons show the inefficiency of the minimal impedance monitoring method. Its underheat rate indicates that based on the ideal test conditions, which means most of the cases have steady compression level and the sticking residual is removed after each test, nearly 40% of the hemostasis processes would require multiple times of rework.

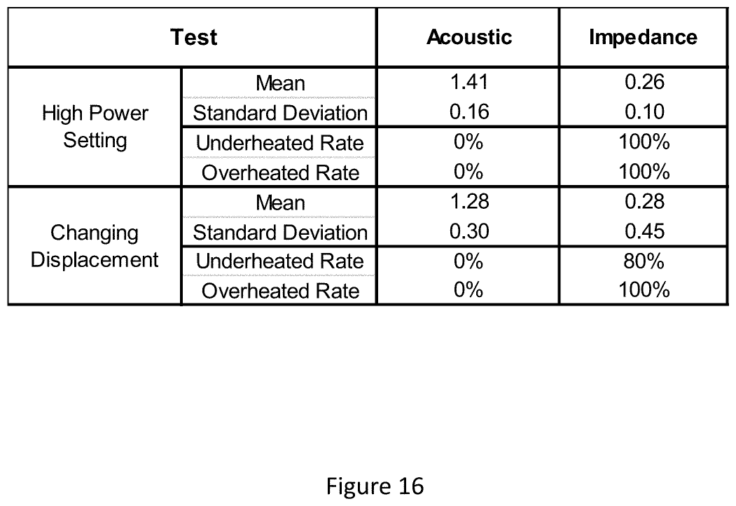

FIG. 16 summarizes the detection results for two surgical operation conditions that were tested in this disclosure. Under the high-power setting and changing displacement scenarios, the minimal impedance monitoring method dramatically overestimated the denatured zone size. The results obtained using the impedance monitoring method indicate that only about a quarter of the total area between the electrodes was denatured, meaning a large diameter vessel would not have been sealed completely using the impedance monitoring method. On the other hand, the sound signal monitoring methods did not fail in any of these tests. The underheated case is defined as cases where Sᵣ is smaller than 1, which means not all the tissue underneath the electrodes was denatured. The underheated case may in some instances be closely related to scenarios where hemostasis was not completely formed when the monitoring method was triggered for completion. The false trigger rate is defined as the percentage of cases which failed to stop the hemostasis process within the acceptable range.

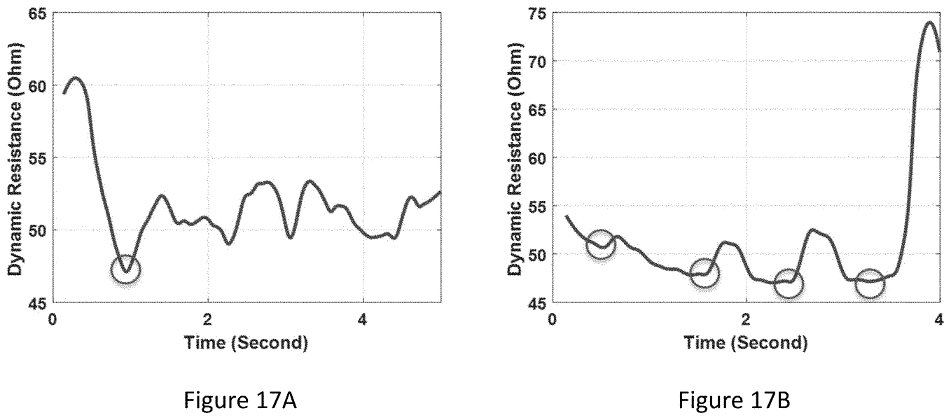

FIG. 17A and FIG. 17B show the typical dynamic resistance plots for the high-power setting and the changing displacement scenarios. The high-power setting makes the water in tissue heat faster, causing some parts of the tissue to reach the boiling temperature faster than others. This will lead to the increase in impedance due to vaporization of the local water content. The impedance monitoring method will thus trigger before the actual completion of the hemostasis process. The same mechanism is in play for the changing displacement scenario, where the displacement between the two electrodes are not kept at the same level throughout the hemostasis process. When the contacting area is changing, the impedance reading will also change even if the tissue has not been denatured yet. These factors, however, will not influence the performance of the sound signal monitoring method due to the nature of sound generation regardless of the power settings and compression conditions applied. The sound signal monitoring method is detecting the sound signal that would be generated only if the temperature inside the tissue reached the same level.

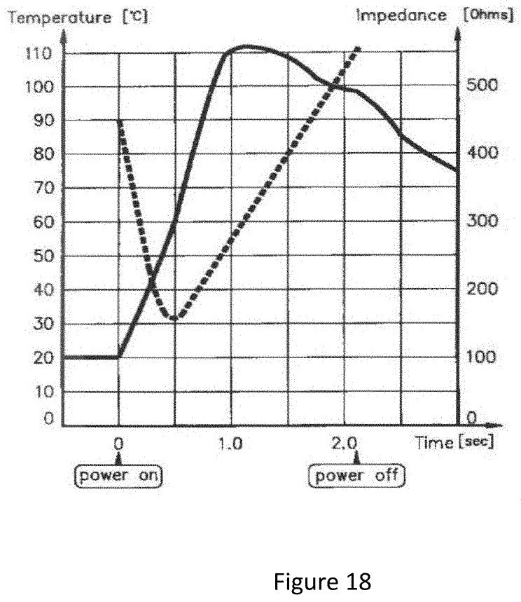

Bergdahl and Vällfors stated that the welding process should be terminated with a delayed duration after the minimal impedance was detected, and FIG. 18 shows a typical impedance plot during the electrosurgical hemostasis process (Bergdahl B et al., Journal of neurosurgery, 1991, 75(1):148-151; Vällfors B et al., Neurosurgical review, 1984, 7(2-3):185-189). This indefinite statement was established based on trial and error tests. With the sound signal monitoring method, the welding cycle should be terminated once the sound signal is triggered, which makes it more robust. The results show that high power settings and varying displacement during the welding process have dramatic influence on the performance of the impedance monitoring method, while the sound signal is more robust because the sizzling sound only starts to be noticeable after the water content in the tissue is heated over the boiling temperature. Bergdahl mentioned that less sticking would be marked on the electrodes with temperature below 120 degrees Celsius, which is higher than the boiling temperature of body water content (Bergdahl B et al., Journal of neurosurgery, 1991, 75(1):148-151). If the power is turned off at the time that the sound signal is triggered, the sticking level is expected to be at an acceptable level. Surgical smoke is another hazardous issue associated with modern electrosurgeries. The sound signal monitoring method stops the heating process before surgical smoke is generated by the vaporization of water in the target tissue.

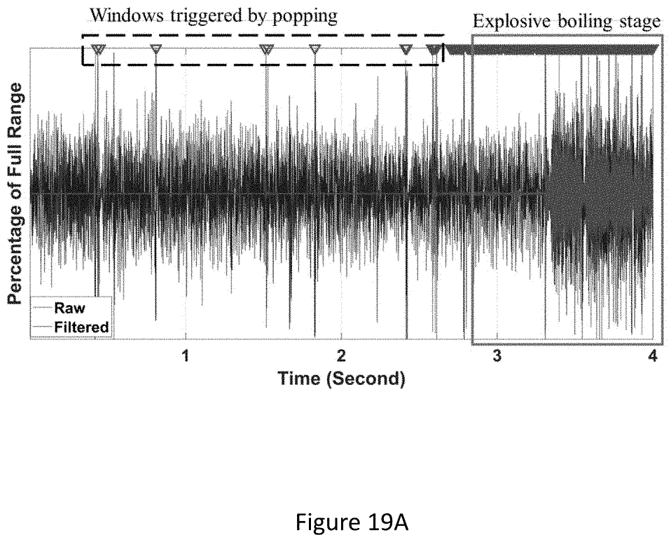

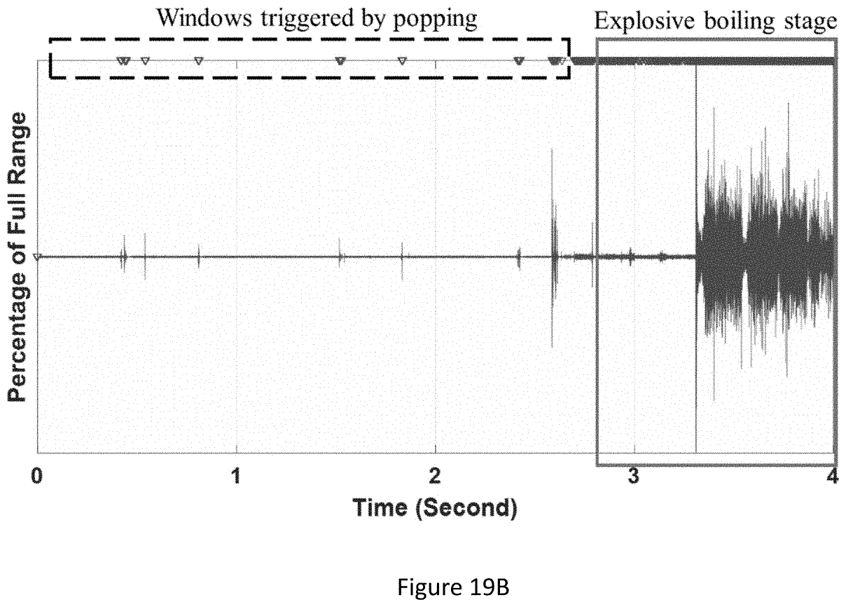

FIG. 19 shows the detection results from the same sample that was manually divided into three stages with the mark of all the triggered window using the sound-based detection method. One advantage shown in this figure is that the sound signal monitoring method can detect the hemostasis completion before human can notice the sizzling sound. The trigger moment of the sound-based monitoring method and manually checked result has 1.02 and 1.35 in projected Sᵣ respectively. The sound signal monitoring method can pick up signals that is inaudible to human ears, especially in a not-so-quiet operating room, and alert surgeons before they can hear the sound.

### Results—Experiment #2

FIG. 19B shows the detection results from the same sample as shown in FIG. 16 that was manually divided into three stages with the mark of all the triggered windows using the sound-based detection method. The explosive boiling stage highlighted in FIG. 19B is machine-detected using the disclosed sound-signal monitoring method. One advantage shown in this figure is that the sound signal monitoring method can detect the hemostasis completion before a human can notice the sizzling sound. The trigger moment of the sound-based monitoring method and manually checked result, which is shown in FIG. 9B, has 1.02 and 1.35 in projected Sᵣ respectively. The disclosed sound signal monitoring method picks up signals that are inaudible to human ears, especially in a not-so-quiet operating room, and alerts surgeons before they can hear the sound.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variation.

What is claimed is:

1. An electrosurgical device comprising:
at least one ablative element;
at least one microphone; and
at least one power lead connected to the at least one ablative element;
wherein the at least one microphone is positioned at a distance from the at least one ablative element.

2. The device of claim 1, wherein the at least one ablative element is selected from the group consisting of: bipolar electrode forceps, unipolar electrodes, laser probes, radiofrequency probes, and microwave probes.

3. The device of claim 2, wherein the bipolar electrode forceps are laparoscopic forceps or tweezer forceps.

4. The device of claim 1, wherein the at least one microphone is selected from the group consisting of: unidirectional microphones, bidirectional microphones, and omnidirectional microphones.

5. The device of claim 1, wherein the at least one microphone is configured to capture a range of acoustic frequencies between about 10 Hz and 24 kHz.

6. The device of claim 1, wherein the distance is between about 1 mm and 20 mm.

7. An electrosurgical system comprising:
an electrosurgical device comprising at least one ablative element, at least one microphone, and at least one power lead connected to the at least one ablative element;
a power source; and
a computing device;
wherein the power source is electrically connected to the computing device and the at least one power lead of the electrosurgical device.

8. The device of claim 7, wherein the system further comprises an oscilloscope electrically connected to the power source and the at least one power lead of the cautery device.

9. The device of claim 7, wherein the system further comprises a current sensor attached to the electronic connection between the power source and the computing device.

10. The device of claim 7, wherein the system further comprises at least one filter attached to the electronic connection between the power source and the computing device, wherein the filter is selected from a low-pass filter, a high-pass filter, and a band-pass filter.

11. A method of controlled electrosurgery, comprising:
positioning an electrosurgery device proximate to a tissue;
positioning an acoustic sensor proximate to the tissue;
measuring a magnitude of an acoustic signal while applying energy to the tissue with the electrosurgical tool;
comparing the magnitude of the acoustic signal to a threshold; and
ceasing to apply energy to the tissue with the electrosurgery device when the magnitude of the acoustic signal exceeds the threshold.

12. The method of claim 11, further comprising applying a conditioning filter to the acoustic signal.

13. The method of claim 11, further comprising calculating the threshold by measuring the acoustic signal for an initial period prior to applying energy to the tissue with the electrosurgery device.

14. The method of claim 13, further comprising calculating the mean and standard deviation of the acoustic signal during the initial period and setting the threshold to N standard deviations above the mean.

15. The method of claim 14, wherein N is at least 6.

16. The method of claim 11, further comprising collecting a window of samples of the acoustic signal of length L samples, calculating the mean, and comparing the mean to the threshold.

17. The method of claim 17, wherein L is at least 100.

18. The method of claim 17, further comprising collecting a plurality of windows of samples of the magnitude of the acoustic signal, and ceasing to apply energy when the mean exceeds the threshold in M consecutive windows of samples.

19. The method of claim 11, further comprising measuring an electrical characteristic of the energy selected from the group consisting of voltage, current, and resistance; and
adjusting the energy applied based on the measured electrical characteristic and the magnitude of the acoustic signal.
